(19) United States
(12) Patent Application Publication
(10) Pub. No.: US 2022/0078538 A1
(43) Pub. Date: Mar. 10, 2022

(54) SYSTEM AND METHOD FOR IMPROVING ACCURACY OF AURAL DETECTION OF SINGING

(71) Applicant: Listen Co., LLC, Scottsdale, AZ (US)

(72) Inventor: Jeffrey Yauck, Scottsdale, AZ (US)

(21) Appl. No.: 17/471,122

(22) Filed: Sep. 9, 2021

Related U.S. Application Data
(60) Provisional application No. 63/076,748, filed on Sep. 10, 2020.

(51) Int. Cl. H04R 1/10

(52) U.S. Cl. CPC H04R 1/1008; H04R 2499/10; H04R 2460/15

(57) ABSTRACT

A system for improving accuracy of self-detection of a voice includes a first cup element having a forward end configured to contact a human face and a rear end configured to be spaced from a human head on which the human face is located such that the first cup element defines a first void configured to receive a first ear of the human head, the first cup element defining an opening to an environment of the system to provide a pathway for sound waves to travel into the first void. The system further includes a second cup element being a mirror image of the first cup element and defining a second void configured to receive a second ear of the human head.

### CROSS-REFERENCE TO RELATED APPLICATIONS

This application claims the benefit of U.S. Provisional Application No. 63/076,748, titled SYSTEM AND METHOD FOR IMPROVING ACCURACY OF AURAL DETECTION OF SINGING and filed Sep. 10, 2020, the entire contents of which is hereby incorporated by reference in its entirety.

### BACKGROUND

### 1. Field

The present disclosure relates to systems and methods for improving aural detection of singing.

### 2. Description of the Related Art

Often times, singers will listen to a recording of themselves singing and notice that what they hear in the recording is not the same as what they hear while practicing. This effect is caused by a combination of sound waves hitting the ear drum at the same time that vibrations from vocal chords travel through the bony skull and again set the ear drum vibrating. As the vibrations travel through the bone they spread out and lower in pitch, giving a false sense of bass. This phenomenon presents difficulties for singers when practicing in both solo and group practice sessions due to the desire to practice in a way to optimize the vocal sound (i.e., acoustic energy generated from a human mouth) in a way that will sound best in a performance setting. However, it is difficult for an individual to optimize their vocal sound when they hear a different version of their voice than others hear. Currently, the only available way for a singer to hear the true sound of her voice is to listen to a recording of her voice. However, this has limitations such as failing to provide real-time feedback. Thus, there is a need in the art for systems and methods for improving accuracy of aural self-detection of singing.

### SUMMARY

Described herein is a system for improving accuracy of aural detection of singing. The system includes a first cup element partially defining a first volume and having a top, a bottom, a first surface extending between the top and the bottom and configured to rest flush against a human face, a second surface extending away from the first surface, and a height from the bottom to the top that is at least two inches such that the first cup element extends at least from a bottom of a first human ear to a top of the first human ear in response to the first human ear being positioned in the first volume. The system further includes a second cup element partially defining a second volume and being a mirror image of the first cup element such that the second cup element extends from at least a bottom of a second human ear that is contralateral to the first human ear to a top of the second human ear in response to the second human ear being positioned in the second volume.

Also disclosed is a system for improving accuracy of self-detection of a voice. The system includes a first cup element having a forward end configured to contact a human face and a rear end configured to be spaced from a human head on which the human face is located such that the first cup element defines a first void configured to receive a first ear of the human head, the first cup element defining an opening to an environment of the system to provide a pathway for sound waves to travel into the first void. The system further includes a second cup element being a mirror image of the first cup element and defining a second void configured to receive a second ear of the human head.

Also disclosed is a method for improving accuracy of self-detection of voice. The method includes obtaining a system having a first cup element having a forward end configured to contact a human face and a rear end configured to be spaced from a human head on which the human face is located such that the first cup element defines a first void configured to receive a first ear of the human head, the first cup element defining an opening to an environment of the system to provide a pathway for sound waves to travel into the first void, and a second cup element being a mirror image of the first cup element and defining a second void configured to receive a second ear of the human head. The method further includes placing the system on a head having the first ear and the second ear such that the first ear is located in the first void and the second ear is located in the second void. The method further includes at least one of singing or speaking while wearing the system.

The foregoing features and elements are to be combined in various combinations without exclusivity, unless expressly indicated otherwise. These features and elements as well as the operation thereof will become more apparent in light of the following description and the accompanying drawings. It should be understood, however, the following description and drawings are intended to be exemplary in nature and non-limiting.

### BRIEF DESCRIPTION OF THE DRAWINGS

FIGS. 1A-1C illustrate a front view (FIG. 1A), a left-side view (FIG. 1B), and a right-side view (FIG. 1C) of a system and an apparatus for improving accuracy of aural self-detection of singing as worn by a user, in accordance with various embodiments of the present disclosure;

FIGS. 2A-2D illustrate a top-down view (FIG. 2A), a front view (FIG. 2B), a perspective view (FIG. 2C), and an enlarged right-side view (FIG. 2D) of a system and an apparatus for improving accuracy of aural self-detection of singing, in accordance with various embodiments of the present disclosure;

FIGS. 3A-3D illustrate a perspective rear view (FIG. 3A), an enlarged left-side view (FIG. 3B), a right-side view as worn by a user (FIG. 3C), and a front-end view as worn by a user of a system and an apparatus for improving accuracy of aural self-detection of singing, in accordance with various embodiments of the present disclosure;

FIG. 4 is a flowchart illustrating a method of making a system and an apparatus for improving accuracy of aural self-detection of singing, in accordance with various embodiments of the present disclosure; and

FIG. 5 is a flowchart illustrating a method of using a system and an apparatus for improving accuracy of aural self-detection of singing, in accordance with various embodiments of the present disclosure.

The subject matter of the present disclosure is particularly pointed out and distinctly claimed in the concluding portion of the specification. A more complete understanding of the present disclosure, however, is best be obtained by referring to the detailed description and claims when considered in connection with the drawing figures, wherein like numerals may denote like elements.

### DETAILED DESCRIPTION

The detailed description of exemplary embodiments herein makes reference to the accompanying drawings, which show exemplary embodiments by way of illustration and their best mode. While these exemplary embodiments are described in sufficient detail to enable those skilled in the art to practice the inventions, it should be understood that other embodiments may be realized and that logical, chemical and mechanical changes may be made without departing from the spirit and scope of the inventions. Thus, the detailed description herein is presented for purposes of illustration only and not of limitation. For example, the steps or blocks recited in any of the method or process descriptions may be executed in any order and are not necessarily limited to the order presented, some steps or blocks that are shown may not be performed, and other steps or blocks that are not shown may be added. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

The present disclosure describes systems, apparatuses, and methods for improving aural detection of singing by an individual during a practice session. As described above, singers and other individuals (such as public speakers) are generally unable to hear the true sound of their voice while speaking or singing due to a combination of sound waves hitting the eardrum at the same time as vibrations traveling through the bony skull. However, the present disclosure provides systems and methods that separate the arrival of the sound waves and the bone vibrations, allowing the individual to hear a more true version of their voice as they speak or sing.

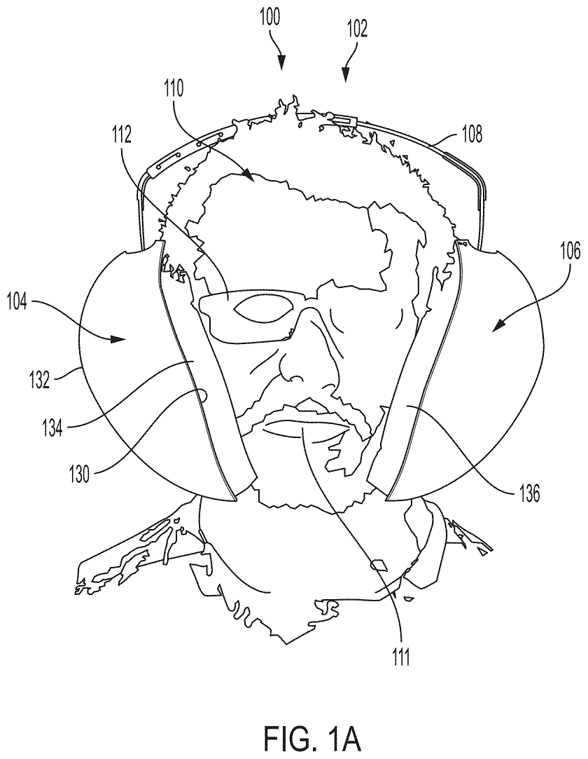

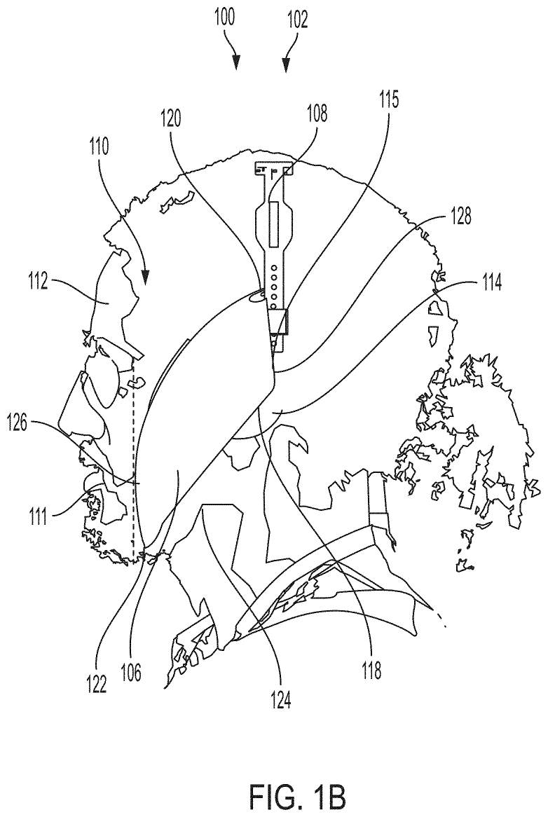

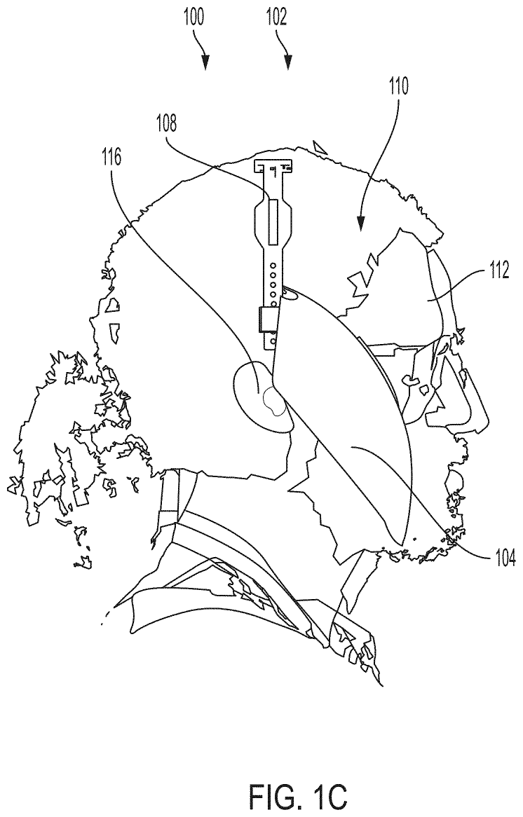

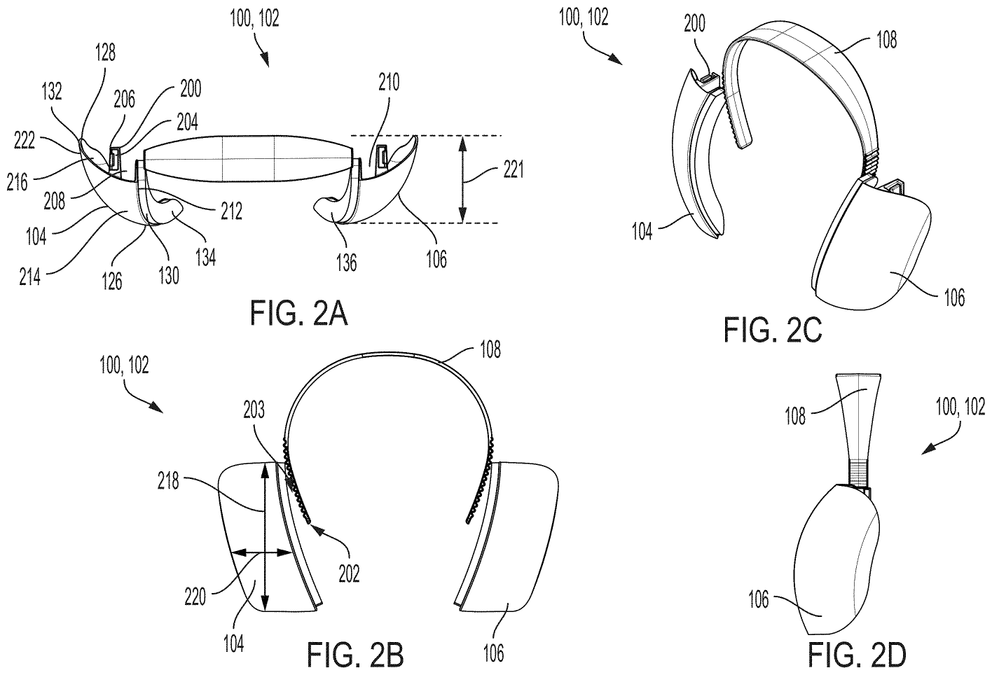

Referring now to FIGS. 1A, 1B, 1C, 2A, 2B, 2C, and 2D, a front view (FIG. 1A), a left-side view (FIG. 1B), and a right-side view (FIG. 1C) of a system 100 and an apparatus 102 for improving accuracy of aural detection of singing are shown as worn by a user, and a top-down view (FIG. 2A), a front view (FIG. 2B), a perspective view (FIG. 2C), and an enlarged right-side view (FIG. 2D) of the system 100 and apparatus 102 without the user are shown. The system 100 and apparatus 102 include a first cup element 104 designed to at least partially sit on a human face 112 and to at least partially interface with, or partially cover, a first, or right, human ear 116. For example, the first cup element 104 may at least partially define a first volume 208 in which the right human ear 116 is positioned. The system 100 and apparatus 102 further include a second cup element 106 designed to at least partially sit on the human face 112 and to at least partially interface with, or partially cover, a second or left human ear 114. For example, the second cup element 106 may at least partially define a second volume 210 in which the left human ear 114 is positioned. In some embodiments, the first cup element 104 may at least one of be a mirror image of the second cup element 106 or include a similar shape as the second cup element 106. The system 100 and apparatus 102 also include a connective element 108 connected to the first cup element 104 and to the second cup element 106, and designed to rest on a human head 110 and to retain the first cup element 104 and the second cup element 106 in place relative to the ears 114, 116. However, one skilled in the art will realize that any other method for retaining the cup elements 104, 106 in place relative to the ears 114, 116 may be used without departing from the scope of the present disclosure. For example, the system 100 and apparatus 102 may include a first frame coupled to the first cup element 104 and a second frame coupled to the second cup element 106 such that the first frame rests on the first human ear 116 and retains the first cup element 104 in place relative to the ear 116, and the second frame rests on the second human ear 114 and retains the second cup element 106 in place relative to the second human ear 114.

In response to the cup elements 104, 106 being positioned over the ears 114, 116 as shown in FIGS. 1A, 1B, and 1C, sound from a human mouth 111 of an individual wearing the system 100 travels around the respective cup elements 104, 106 prior to reaching the ears 114, 116. This delay results in the ears 114, 116 perceiving the sound less as an individual typically perceives their own voice (e.g., because the arrival of the sound waves and the vibrations through the bone are separated and not simultaneous) and more as others perceive their voice. This effect is provided by positioning the ears 114, 116 within the volumes 208, 210 and providing an opening at the back of the cup elements 104, 106 (i.e., between the head 110 and the cup elements 104, 106 at the back of the cup elements 104, 106) through which sound waves may travel. Thus, the system 100 and apparatus 102 provide an invaluable practice tool for any amateur and professional singers who wish to more accurately perceive the sound of their own singing. Similarly, the system 100 and apparatus 102 may be used by a public speaker wishing to improve delivery of speech.

The connective element 108 may be coupled to the first cup element 104 and the second cup element 106 in any of a variety of manners. For example and as shown, each of the cup elements 104, 106 may include a fastener 200 (e.g., a female fastener portion) designed to interface with a fastener 202 (e.g., a male fastener portion) of the connective element 108. The fastener 200 may be removably coupled or moveably coupled to the fastener 202 in order to allow the cup elements 104, 106 to move relative to the connective element 108 and, thus, to move relative to the ears 114, 116. In particular, the fastener 200 of the cup element 104 may include a slot 204 in which a portion of the connective element 108 may be received and a latch 206. The connective element 108 may include a plurality of teeth 203. An end of the connective element 108 may be inserted into the slot 204 until the cup element 104 is positioned at a desired location such that the interface of one of the teeth 203 in contact with the latch 206 may resist further movement of the first cup element 104 relative to the connective element 108. However, one skilled in the art will realize that any other fastener designs may be used to couple the cup elements 104, 106 to the connective element 108. For example, the cup elements 104, 106 may be welded or formed integral with the connective element 108, a press-fit connection may be used between the cup elements 104, 106 and the connective element, the connective element 108 may fail to be present and may be replaced with separate frames each coupled to one of the cup elements 104, 106, hook and loop fasteners or screw threading may be used as the fasteners 200, 202, or the like.

The cup elements 104, 106 may be mirror images of each other. That is, the cup elements 104, 106 may be the same except for having a reverse structure, as would be viewed in a mirror. The cup elements 104, 106 may each include a top 120, a bottom 122, a forward end 126, a rear end 128, an inner end 130, and an outer end 132.

The following paragraphs describe desirable characteristics of the cup elements 104, 106 that provide optimal acoustic characteristics, and later paragraphs describe exemplary dimensions for the cup elements 104, 106. The desirable characteristics may correspond to dimensions of a human face or head. In that regard, the characteristics may be selected based on an average set of facial features, two apparatuses 102 may be made available such that a first corresponds to average male facial features and a second corresponds to average female facial features, characteristics may be selected based on a certain percentile (e.g., 80^{th} percentile, 90^{th} percentile, 95^{th} percentile, 99^{th} percentile, or the like) such that the dimensions are sufficient to provide desirable acoustics to a relatively large proportion of the population, or the like. However, one skilled in the art will realize that the disclosure is not limited by the desirable characteristics described herein.

The characteristics may utilize directional language which may correspond to directional descriptors conventionally utilized when referencing a human head. For example, forward or front may refer to a direction in which eyes typically face (also a direction in which a nose extends away from the face), and back or rear may refer to a direction opposite the forward or front direction. Up or top may refer to a direction above a head when the user is in a standard seated or standing position (also a direction parallel to a line from a center of a mouth towards a nose or from edges of the mouth towards eyes), and down or bottom may refer to a direction opposite the up or top direction.

In some embodiments, the forward end 126 (i.e., a portion or edge of the cup elements 104, 106 that is nearest to a nose of the head 110 when properly worn on the head 110) may be located forward relative to the ears 114, 116 (e.g., the forward end 126 may be positioned closer to the nose than the ears 114, 116). In some embodiments, the forward end 126 may remain forward relative to the ears 114, 116 for a distance that is at least as great as a height of the ears 114, 116. The rear end 128 of the cup elements 104, 106 (e.g., a portion or edge of the cup elements 104, 106 that nearest to a back on the head 110 in response to being properly worn on the head 110) may be designed to extend at least to a middle of an ear canal 118 of the ears 114, 116, such that the cup elements 104, 106 may extend from a location forward (i.e., towards a nose) of the front of the ears 114, 116 to at least a middle of the ear canal 118. The middle of the ear canal 118 may refer to a midpoint of the ear canal 118 in a direction between a front of the head 110 and a back of the head 110. In some embodiments, the rear end 128 may extend farther towards a back of the head 110 than the middle of the ear canal 118. In some embodiments, the rear end 128 may end before the middle of the ear canal 118.

In some embodiments, the rear end 128 may be spaced apart from the head 110 by a distance (e.g., 0.5 inches (12.7 millimeters (mm)), 1 inch (25.4 mm), 2 inches (50.8 mm), or the like). Such spacing may create a gap between the cup elements 104, 106 and the head 110 such that an opening exists between the cup elements 104, 106 and the environment to allow sound waves to clearly reach the eardrum without traveling through materials. Such an opening results in a desirable wavefront shape reaching the eardrum, providing a relatively accurate representation of the user's voice.

In some embodiments, the bottom 122 of the cup elements 104, 106 (e.g., a portion or edge located nearest shoulders when worn on the head 110) may extend to or beyond (i.e., below, or towards shoulders from) a jawline 124 of the human head 110. The jawline may refer to a bottom ridge of a jaw of the user, may refer to a line extending straight backwards from a bottom of a chin of the user, or the like. In some embodiments, the top 120 of the cup elements 104, 106 (e.g., a portion or edge located nearest a top of the head 110, or farthest from a neck, when worn on the head 110) may extend to or above a top 115 of the ear 114.

The above-described characteristics of the cup elements 104, 106 (e.g., extending from a location forward of the front of the ear to at least the middle of the ear canal 118 and extending from at least the jawline 124 to the top 115 of the ear 114) may sufficiently delay sound waves from traveling from the mouth 111 to the eardrums that the sound waves and bone vibrations reach the eardrums at different times, thus allowing the user to hear a truer representation of her singing or speech. These characteristics may further provide aesthetic appeal. Additional characteristics described below may further improve the accuracy of the sound, and may provide further aesthetic appeal.

In some embodiments, the cup elements 104, 106 may curve or bow forward (i.e., towards the forward end 126) between the top 120 and the bottom 122. That is, a forward edge of the cup elements 104, 106 (i.e., an edge between the top 120 and bottom 122 that includes the forward end 126) may curve or bow in a forward direction (i.e., the edge may curve or bow towards a nose). Such curvature may be for aesthetic purposes, acoustic purposes, or the like. For example, this curvature may provide increased distance in which sound travels prior to reaching the ear 114, thus improving acoustic response of the apparatus 102. This curvature may also result in a desirable wavefront shape of the sound waves hitting the eardrums, further improving acoustics of the received singing or speech.

The outer end 132 may include a portion or edge of the cup elements 104, 106 that is located farthest away from the head 110 when worn as shown. In some embodiments, the cup elements 104, 106 may curve or bow outwards (i.e., towards the outer end 132 and away from the head 110) between the top 120 and the bottom 122. This curvature may likewise be for aesthetic purposes, acoustic purposes, or the like. For example, this curvature may also at least one of provide increased distance in which the sound travels prior to reaching the ear 114 or result in a desirable wavefront shape hitting the eardrums, thus improving acoustic response of the apparatus 102.

The cup elements 104, 106 include an inner edge or surface 212 (which includes the inner end 130 and is nearest to the face) designed to rest on, or contact, a face when the cup elements 104, 106 are worn. The cup elements may further include an outer end 132 that is farthest from the face when worn as shown. The inner edge or surface 212 may be the nearest edge or surface to the head 110. In some embodiments, the apparatus 102 may further include a first padding 134 and a second padding 136 coupled to a portion or all of the surface 212. The first padding 134 may be permanently or removably coupled to the first cup element 104, and the second padding 136 may be permanently or removably coupled to the second cup element 106. In that regard, the first padding 134 may rest between the first cup element 104 and the face 112, and the second padding 136 may rest between the second cup element 106 and the face 112. The first padding 134 and the second padding 136 may provide a cushion between the cup elements 104, 106 and the face 112 in order to make the apparatus 102 more comfortable for the user. In that regard, it is desirable for the padding 134, 136 to be relatively soft (e.g., squishy) so it is comfortable when adjacent to skin or hair, to be hypoallergenic, and to have a relatively smooth surface so as to avoid irritating skin should the padding 134, 136 rub against the skin.

In some embodiments, the first padding 134 and the second padding 136 may include a material that resists the passage of sound waves therethrough. For example, the paddings 134, 136 may be formed from or include a material such as cloth (any cloth such as chiffon, cotton, or the like), leather (from any animals or synthetic leather), or acoustic foam (e.g., any polyurethane, polyether, polyester, melamine foam, or the like) in order to absorb sound waves, a harder material such as a plastic (e.g., polyethylene terephthalate (PET), high-density polyethylene (HDPE), polyvinyl chloride (PVC or vinyl), low-density polyethylene (LDPE), polypropylene (PP), polystyrene (PS or Styrofoam) or the like) or a rubber (natural or synthetic such as neoprene, silicone, nitrile, butyl, fluorosilicone, or the like) to reflect the sound waves, or the like.

The cup elements 104, 106 may likewise be formed from or include a material to either reflect sound waves or absorb sound waves. For example, the cup elements 104, 106 may include a plastic (e.g., PET, HDPE, PVC, LDPE, PP, PS, or the like), an acoustic foam (e.g., any polyurethane, polyether, polyester, melamine foam, or the like), a rubber (natural or synthetic such as neoprene, silicone, nitrile, butyl, fluorosinicone, or the like), or any other material. As another example, the cup elements 104, 106 may each include an acoustic foam core that is coated in a plastic (e.g., any plastic material, polytetrafluoroethylene (PTFE), or the like). In some embodiments, the cup elements 104, 106 and the connective element 108 may be formed from a relatively lightweight material. In some embodiments, the cup elements 104, 106 may include a denser material that is formed to have voids (e.g., formed into a honeycomb structure) which may or may not be coated. This advantageously increases comfort of the apparatus 102. In some embodiments, a tradeoff may be made between reflection or absorbtion of sound waves and the weight of the apparatus 102, as an inversely proportional relationship may exist between reflection/absorption and weight.

The cup elements 104, 106 may each define or include a first surface 212, a second surface 214, and a third surface 216. The first surface 212 may be oriented substantially parallel to a side of the head 110. Where used in this context, “substantially parallel” indicates that the first surface 212 may be parallel to the side of the head 110 or may form any angle relative to the head 110 that is between zero degrees and 45 degrees. For example, “substantially parallel” may indicate that the first surface 212 forms an angle that is less than or equal to 10 degrees with the head 110, less than or equal to 20 degrees with the head 110, less than or equal to 30 degrees with the head 110, or the like.

The first surface 212 may directly or indirectly lie flush with the face 112 (i.e., may rest on, or contact, the face 112) at a location in front (i.e., forward of) of the ears 114, 116. When the padding 134 is utilized, the padding 134 may be permanently or removably coupled to the first surface. For example, a fastener (e.g., screw, bolt, snap-fit connector, hook-and-loop fasteners, or the like) may be used to couple the padding 134 to the first surface 212. As another example, an adhesive may be used to couple the padding 134 to the first surface 212. The padding 134 may rest between the face 112 and the first surface 212. In that regard, the first surface 212 may provide a surface upon which the first cup element 104 may be positioned on the face 112. The first surface 212 may be relatively flat, may have a curvature designed to mimic a curvature of the face 112, or may include a combination of straight portions and curved portions.

The second surface 214 may extend away from the first surface 212. Accordingly, the second surface 214 may extend away from the face 112 at a location in front of the ear 114. This extension of the first cup element 104 away from the face 112 at least one of increases a distance which sound from the mouth 111 travels before reaching the ear 114 or results in a desirable wavefront shape hitting the eardrum, thus improving accuracy of the sound as heard by the ear 114. A transition from the first surface 212 to the second surface 214 may be abrupt (i.e., angled) or may be curved. In addition, the second surface 214 may be relatively straight, may have a curvature, or may include a combination of straight portions and curved portions. For example and as shown, the second surface 214 may curve rearward as it extends away from the first surface 212. The curvature of the second surface 214 may increase both acoustic response of the cup element 104 as well as aesthetics of the apparatus 102.

The third surface 216 may be extend towards the back of the head 110 from the second surface 214. In some embodiments, the third surface 216 may be substantially parallel to the first surface 212. In some embodiments, the third surface 216 may be an extension of the second surface such that an abrupt transition between the second and third surfaces 214, 216 fails to exist. In some embodiments, the transition between the second and third surfaces 214, 216 may be abrupt and may include curvature, an angle, or the like. The third surface 216 may define the rear end 128 and may extend to or beyond the middle of the ear canal 118. In some embodiments, a transition 222 from the second surface 214 to the third surface 216 may be straight (i.e., angled) or curved. A curved transition 222 may increase both acoustic response of the cup element 104 as well as aesthetics of the apparatus 102.

A gap or void may exist between the second surface 214 and the head 110, and between the third surface 216 and the head 110. Such a gap or void allows sound waves to directly reach the eardrums without traveling through a material, allowing the audible sound to be more realistic than if the sound were required to travel through the cup elements 104, 106.

In some embodiments, the top 120 may be located closer to the rear end 128 than the bottom 122, and the bottom 122 may be located closer to the forward end 126 than the top 120. That is, the cup elements 104, 106 may be swept rearward towards the top 120. This shape allows the apparatus 102 to be formed using less material (e.g., because it can still cover the desirable portions of the ears 114, 116), providing benefits such as a lighter and more comfortable apparatus, a reduced cost of manufacture, a more environmentally friendly product, or the like.

The cup elements 104, 106 may each have a height 218 (i.e., from top 120 to bottom 122), a width 220 (i.e., from the inner end 130 to the outer end 132), and a length 221 (i.e., from the forward end 126 to the rear end 128).

The height 218 may be sufficiently large so as to provide for extension of the cup elements 104, 106 from at least the top 115 of the ear 114 to the jawline 124 (e.g., for an average human head, for 90 percent of human heads, for 99 percent of human heads, or the like). For example, the height 218 may be between 1 inch and 10 inches (25.4 millimeters (mm) and 254 mm), between 2 inches and 8 inches (50.8 mm and 203.2 mm), between 3 inches and 5 inches (76.2 mm and 127 mm), or about 4 inches (101.6 mm). Where used in this context, “about” refers to the referenced value plus or minus 20 percent of the referenced value.

The width 220 may be sufficiently large so as to cause the outer end 132 of the cup elements 104, 106 to extend beyond an outer portion of the ears 114, 116. In some embodiments, it is desirable for the outer end of the cup elements 104, 106 to extend outward by a sufficiently great distance that the cup elements 104, 106 fail to contact the ears 114, 116. For example, the width 220 may be between 0.5 inches and 6 inches (12.7 mm and 152.4 mm), between 0.5 inches and 4 inches (12.7 mm and 101.6 mm), between 1 inch and 3 inches (25.4 mm and 76.2 mm), or about 1.5 inches (38.1 mm).

The length 221 may be sufficiently large so as to cause the forward end 126 to rest in front of the ears 114, 116 and to cause the rear end 128 to extend to or beyond the middle of the ear canal 118. It is desirable for the forward end 126 to be located forward from an entire forward edge of the ear 114, 116 to achieve desirable acoustic quality. For example, the length 221 may be between 0.5 inches and 6 inches (12.7 mm and 152.4 mm), between 1 inch and 5 inches (25.4 mm and 127 mm), between 1 inch and 4 inches (25.4 mm and 101.6 mm), or about 2 inches.

In some embodiments, the cup elements 104, 106 may be adjustable and, thus, may have adjustable dimensions. For example, at least one of the height 218, the width 220, or the length 221 may be adjustable. For example, the cup elements 104, 106 may each include multiple parts that may move relative to each other, allowing the user to configure the cup elements 104, 106 to have desirable dimensions for specific features of the user (e.g., based on ear size, ear position, or the like). The cup elements 104, 106 may include any features that facilitate such adjustment. For example, the cup elements 104, 106 may each include multiple portions connected together using adjustable connectors such as screw threads, snap connectors with multiple snap positions, clamps that allow relative movement of the portions when loosened and resist such relative movement when tightened, or the like.

In some embodiments, the cup elements may lack adjustability. In some embodiments, multiple versions of the system 100 and apparatus 102 may be available for use, each version having different dimensions. In some embodiments, different versions may be available that each include different materials, different combinations of materials, different decorations (e.g., colors, patterns, or the like), or any different configurations.

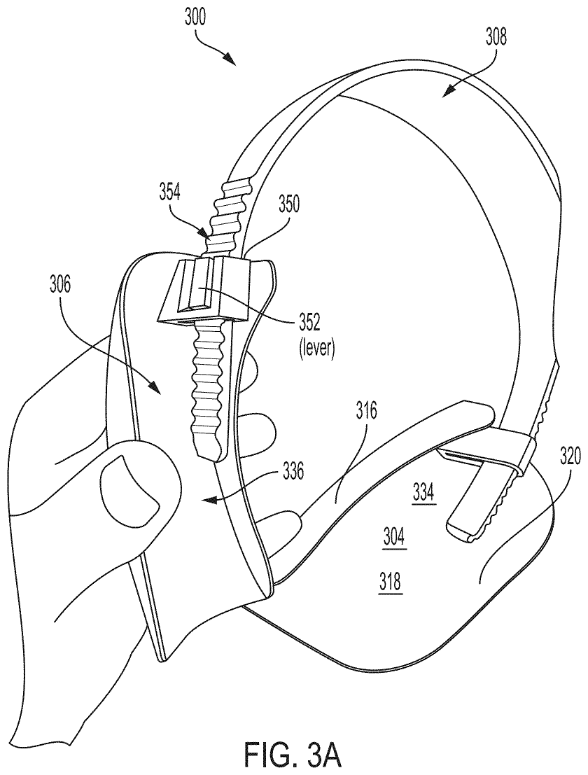

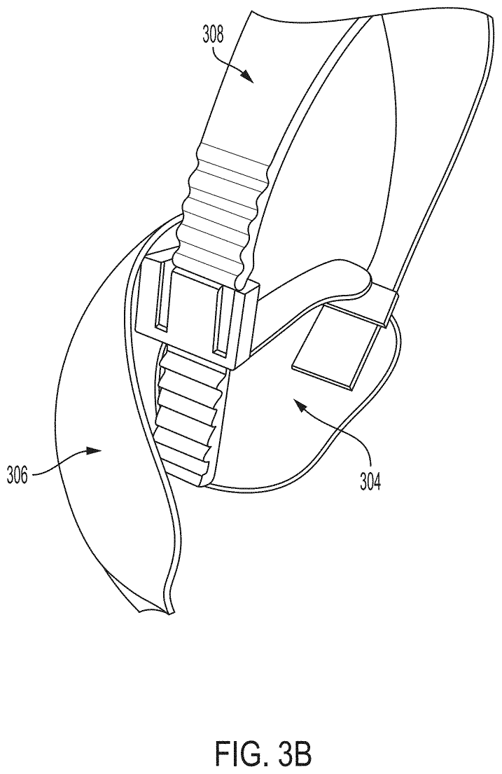

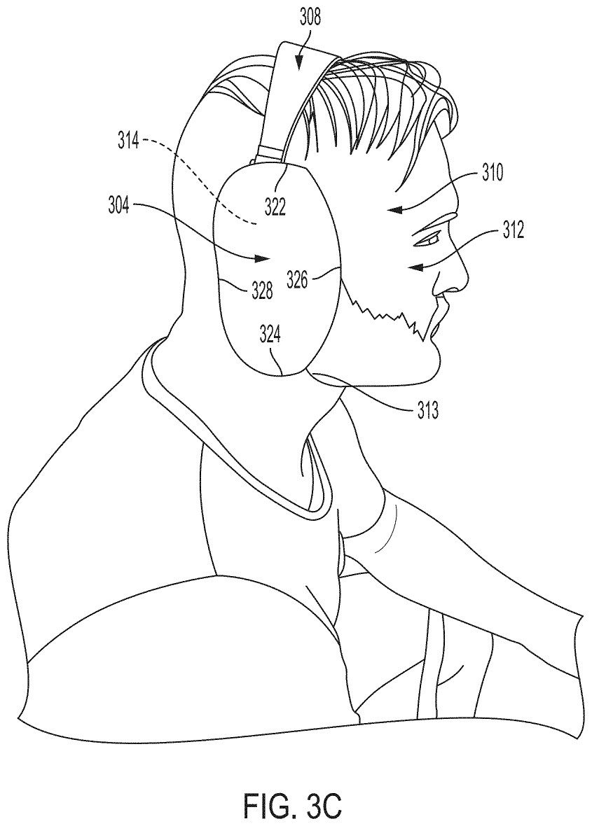

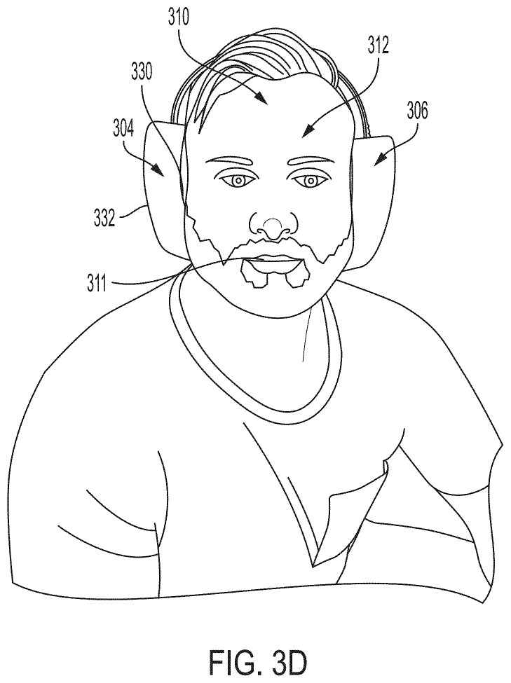

A system and apparatus similar to the system 100 and apparatus 102 of FIGS. 1A-2D may include similar features having different shapes than those shown in FIGS. 1A-2D. For example and referring to FIGS. 3A, 3B, 3C, and 3D, a perspective rear view (FIG. 3A), an enlarged left-side view (FIG. 3B), a right-side view as worn by a user (FIG. 3C), and a front-end view as worn by a user (FIG. 3D) of a system 300 having similar features as the system 100 shown in FIGS. 1A-2D are shown. The system 300 may include at least one of different shaped elements, different dimensions, or other different features than the system 100.

In particular, the system 300 may include a first cup element 304, a second cup element 306, and a connective element 308. The first cup element 304 and the second cup element 306 may each define a volume that is designed to at least partially enclose ears of a user therein. The cup elements 304, 306 may each include a first surface 316, a second surface 318, and a third surface 320. The first surface 316 is designed to rest on a face 312 of the user. The second surface 318 may extend away from the first surface 316 in a direction substantially perpendicular to the first surface 316 (i.e., the second surface 318 may extend outward from the first surface 316 and, thus, the face 312). The third surface may extend from the second surface 318 in a direction that is substantially parallel to the first surface 316. The cup elements 304, 306 may each include a forward end 326, a rear end 328, a top 322, a bottom 324, an inner end 330, and an outer end 332. As shown, the cup elements 304, 306 may each at least partially enclose ears of the user, and may do so using different shapes than the respective cup elements 104, 106 of the system 100 of FIGS. 1A-2D. However, the system 300 may still provide desirable acoustics so long as the cup elements 304, 306 extend at least from a top of an ear 314 to a jawline 313 of the user, and from a front of the ear 314 to at least a middle of an ear canal of the ear 314. In some embodiments, the cup elements 304, 306 may extend from the front of the ear 314 to at least a rear end of the ear 314.

For example, the cup elements 304, 306 may each be kidney shaped when viewed from a side (e.g., the view shown in FIG. 3C). The forward end 326 may be curved and may bow forward (i.e., towards a nose) between the top 322 and the bottom 324. The rear end 328 may have a similar curve as the forward end 326 and may likewise be curved and bow forward between the top 322 and the bottom 324.

The first surface 316 may be curved and may bow outward (i.e., away from the face) between the top 322 and the bottom 324. This curvature may allow the cup elements 304, 306 to better rest against the face.

As with the connective element 108 of FIGS. 1A-2D, the connective element 308 may rest on a head 310 of the user and may be coupled to the first cup element 304 and to the second cup element 306. The connective element 308 may resist separation of the cup elements 304, 306 from the connective element 308. The connection between the connective element 308 and the cup elements 304, 306 may be adjustable such that the cup elements 304, 306 may be repositioned relative to ears of the user to optimize acoustic response of the system 300. In particular, the cup elements 304, 306 may each include or define a slot 350 with a lever 352 attached thereto, and the connective element 308 may include a plurality of teeth 354 on each end. The teeth 354 may extend through the slot 350 such that the lever 352 resists movement of the teeth 354 through the slot 350 (the lever 352 may be urged away from the teeth 354 to facilitate movement of the teeth 354 through the slot 350). This adjustable engagement between the lever 352 and teeth 354 allows the cup elements 304, 306 to be raised and lowered, and then retained in place relative to the connective element 308 to hold the cup elements 304, 306 in a desired location.

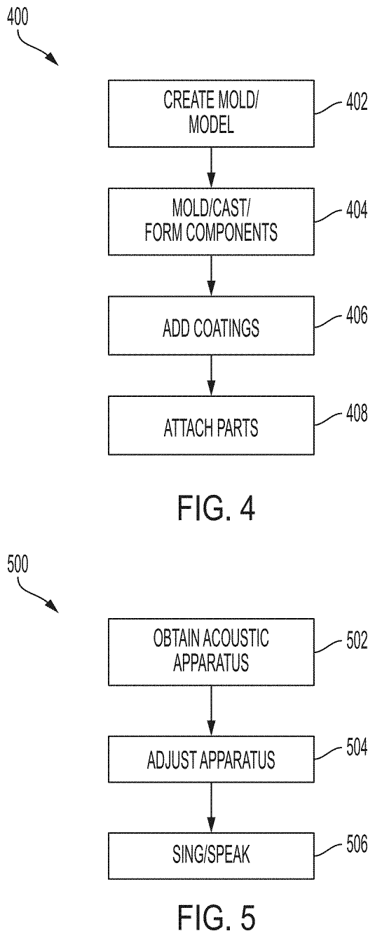

Referring now to FIG. 4, a method 400 for forming a system or apparatus similar to those shown in FIGS. 1A-3D is shown. The system/apparatus may be formed using any known method such as additive manufacturing, molding, casting, or the like. The method 400 may include additional blocks other than those shown in FIG. 4, may lack some blocks shown in FIG. 4, or the like without departing from the scope of the present disclosure.

In block 402, a mold, a cast, a model, or the like of the components of the system/apparatus may be created. For example, desirable dimensions and shapes of the cup elements and connective element may be selected, and a mold, cast, model (e.g., a three-dimensional model created using software), or the like may be created based on the desirable dimensions and shapes.

In block 404, the cup elements and connective element may be created using any known method. If padding is to be utilized the padding may likewise be formed in block 404. For example, the elements may be created using injection molding, rotational molding, extrusion molding, blow molding, reaction injection molding, vacuum casting, thermoforming, compression molding, additive manufacturing, or the like. In some embodiments, the elements may be formed using different methods. For example, the cup elements may be formed using additive manufacturing and the connective element may be formed using extrusion molding.

In block 406, any coatings to be applied to the elements may be added. For example, the cup elements may include a honeycomb core that is coated in a plastic. In that regard, the plastic coating may be added to the core. The coating may be applied in any known manner such as dip coating, brushing, roll coating, spraying, spin coating, flow coating, or the like.

In block 408, any parts, or elements, may be coupled together. For example, any padding may be coupled to the inner surfaces of the cup elements, and the cup elements may be coupled to the connective element. Such attachment may include any attachment means such as adhesive, fasteners, snap connections, or the like.

Referring to FIG. 5, a method 500 for singing or speaking such that the singer/speaker hears a true representation of their own voice is shown. The method 500 may be used by any individual for any purpose such as to practice singing or public speaking, to better gauge sound while performing, or the like.

In block 502, an individual may obtain an acoustic system or apparatus such as those described above having two cup elements and a connective element. The individual may obtain such system or apparatus in any manner such as manufacturing or otherwise forming the system/apparatus, purchasing the system/apparatus, or the like.

In block 504, if the system/apparatus is adjustable (e.g., if the dimensions of the cup elements may be adjusted, or if the position of the cup elements relative to the connective element may be adjusted) then the user may adjust the various features of the system/apparatus. The user may make such adjustments to cause the cup elements to extend from a location at or above the ear to a location at or below the jawline, to extend from a location at or forward from a forward end of the ear to a location at or back from a middle of an ear canal of the ear. Such a configuration may provide optimal acoustics to the individual.

In block 506, the individual may at least one of sing or speak while wearing the system/apparatus. Such use of the system/apparatus delays passage of the sound waves from the mouth to the ear drums, thus separating arrival of the sound waves from arrival of the bony vibrations and allowing the user to hear a more true representation of his or her voice. Such use of the system may also cause a desirable wavefront shape of the sound wave to hit the ear, further improving accuracy of the singing or speaking.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the inventions. The scope of the invention is accordingly to be limited by nothing other than the appended claims, in which reference to an element in the singular is not intended to mean “one and only one” unless explicitly so stated, but rather “one or more.” Moreover, where a phrase similar to “at least one of A, B, or C” (and a phrase similar to “at least one of A, B, and C”) is used in the specification and claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching is used throughout the figures to denote different parts but not necessarily to denote the same or different materials.

Systems, methods and apparatus are provided herein. In the detailed description herein, references to “one embodiment,” “an embodiment,” “various embodiments,” “some embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A system for improving accuracy of aural detection of singing, the system comprising:
a first cup element partially defining a first volume and having a top, a bottom, a first surface extending between the top and the bottom and configured to rest flush against a human face, a second surface extending away from the first surface, and a height from the bottom to the top that is at least two inches such that the first cup element extends at least from a bottom of a first human ear to a top of the first human ear in response to the first human ear being positioned in the first volume; and
a second cup element partially defining a second volume and being a mirror image of the first cup element such that the second cup element extends from at least a bottom of a second human ear that is contralateral to the first human ear to a top of the second human ear in response to the second human ear being positioned in the second volume.

2. The system of claim 1 wherein the first cup element includes a third surface extending away from the second surface in a direction substantially parallel to the first surface.

3. The system of claim 2 wherein the third surface is configured to extend at least to a middle of an ear canal of the first human ear in response to the system being worn on a human head.

4. The system of claim 2 wherein the third surface is an extension of the second surface and wherein a transition from the second surface to the third surface is curved.

5. The system of claim 1 further comprising a connective element configured to extend from the first cup element to the second cup element and to rest on a top of a human head and to retain the first cup element and the second cup element in place relative to the first human ear and the second human ear.

6. The system of claim 5 wherein a first connection between the connective element and the first cup element and a second connection between the connective element and the second cup element is adjustable to facilitate adjustment of the first cup element and the second cup element relative to the first human ear and the second human ear.

7. The system of claim 1 wherein the first cup element and the second cup element are formed from a plastic material.

8. The system of claim 1 wherein the first cup element and the second cup element are configured to cause acoustic energy from a mouth of a human having the first human ear and the second human ear to propagate around the first cup element and the second cup element to replicate the acoustic energy in the first human ear and in the second human ear as heard by a different human.

9. The system of claim 1 wherein the first cup element has a forward end and a rear end, and wherein the first cup element curves towards the forward end between the top and the bottom.

10. The system of claim 9 wherein the first cup element has an inner end and an outer end, and wherein the first cup element curves towards the outer end between the top and the bottom.

11. The system of claim 1 wherein the bottom is configured to extend to or below a human jawline and the top is configured to extend to or above a top of the first human ear in response to the system being worn on a human head.

12. The system of claim 1 further comprising a first padding coupled to the first surface of the first cup element and configured to provide a cushion between the first surface and the human face.

13. The system of claim 1 wherein the first cup element at least partially defines an opening to the first volume to facilitate travel of sound waves into the first volume from an environment of the first cup element.

14. A system for improving accuracy of self-detection of a voice, the system comprising:
a first cup element having a forward end configured to contact a human face and a rear end configured to be spaced from a human head on which the human face is located such that the first cup element defines a first void configured to receive a first ear of the human head, the first cup element defining an opening to an environment of the system to provide a pathway for sound waves to travel into the first void; and
a second cup element being a mirror image of the first cup element and defining a second void configured to receive a second ear of the human head.

15. The system of claim 14 further comprising a connective element configured to be coupled to the first cup element and the second cup element and to resist separation of the first cup element and the second cup element from the connective element.

16. The system of claim 14 wherein the forward end of the first cup element extends farther inward than the rear end of the first cup element such that the opening is defined between the rear end and the human head.

17. The system of claim 16 wherein a height of the first cup element from a top of the first cup element to a bottom of the first cup element is at least two inches, and a length of the first cup element from the forward end to the rear end is at least 1 inch.

18. The system of claim 14 wherein the first cup element bows forward between a top of the first cup element and a bottom of the first cup element, and further bows outward between the forward end and the rear end.

19. The system of claim 14 further comprising a first padding coupled proximate to the forward end of the first cup element and configured to provide a cushion between the first cup element and the human face, and a second padding coupled proximate to the forward end of the second cup element and configured to provide a cushion between the second cup element and the human face.

20. A method for improving accuracy of self-detection of voice, the method comprising:
obtaining a system having:
a first cup element having a forward end configured to contact a human face and a rear end configured to be spaced from a human head on which the human face is located such that the first cup element defines a first void configured to receive a first ear of the human head, the first cup element defining an opening to an environment of the system to provide a pathway for sound waves to travel into the first void, and
a second cup element being a mirror image of the first cup element and defining a second void configured to receive a second ear of the human head;
placing the system on a head having the first ear and the second ear such that the first ear is located in the first void and the second ear is located in the second void; and
at least one of singing or speaking while wearing the system.
